(19) United States
(12) Patent Application Publication
(10) Pub. No.: US 2026/0207172 A1
(43) Pub. Date: Jul. 23, 2026

(54) ULTRASONIC CONTRAST IMAGING DEVICE AND METHOD BASED ON ULTRAFAST PLANE WAVE

(71) Applicant: ESONIC MEDICAL TECHNOLOGY (BEIJING) CO., LTD., Beijing (CN)

(72) Inventors: Zhenyao Wen, Beijing (CN); Yong Ding, Beijing (CN); Jacques Souquet, Beijing (CN)

(21) Appl. No.: 19/117,132

(22) PCT Filed: Jul. 24, 2024
(86) PCT No.: PCT/CN2024/107175

§ 371 Date: Mar. 31, 2025

(30) Foreign Application Priority Data
Sep. 18, 2023 (CN) 202311203841.8

(51) Int. Cl. A61B 8/00

(52) U.S. Cl. CPC A61B 8/481; A61B 8/5207

(57) ABSTRACT

Disclosed are an ultrasonic contrast imaging device and a method based on the ultrafast plane wave, including: acquiring a plane wave sequence with an emission waveform of [k, 1, 1−k, −1]; separately emitting corresponding ultrasonic plane waves according to an emission order based on a preset emission end; and sequentially collecting echo signals corresponding to different ultrasonic plane waves after a target duration; performing signal processing on the collected echo signals and storing the processed echo signals; transmitting the stored echo signals to the master computer; superimposing different echo signals according to weights of different echo signals based on the master computer to obtain a sum signal; and performing beam synthesis processing on the sum signal to obtain a beam synthesis signal; and performing a modulus calculation and a pseudo-color mapping display for the beam synthesis signal to obtain an ultrasonic contrast image at a target moment.

### TECHNICAL FIELD

The present disclosure relates to the technical field of medical imaging, and particularly relates to an ultrasonic contrast imaging device and method based on ultrafast plane wave.

### BACKGROUND ART

At present, ultrasonic contrast is a very common medical imaging technology, which provides richer image information by utilizing the interaction of ultrasonic waves with specific types of contrast agents. The ultrasonic contrast has the advantages of being non-invasive, radiation-free, and easy to operate, so as to be widely used in clinical diagnosis and lesion monitoring.

However, the current ultrasonic contrast imaging faces some challenges, including the following aspects.
1. Frame frequency limitation: the traditional ultrasonic contrast method improves the sensitivity of contrast imaging by using contrast pulse sequencing (CPS) emission technology based on the amplitude modulation. However, the frame frequency is reduced, which limits the improvement of the frame frequency for the clinical device when it needs to display real-time B-mode images simultaneously to observe tissue structure information.
2. Front-end resource consumption: the current soft beam synthesis method consumes large resources in front-end processing due to the involvement of the weighted superposition and combination processing of multiple emission waveforms.
3. Limitations of time information: the time measurement in the ultrasonic contrast is of critical importance for aspects such as mobility assessment and cardiac function assessment; however, the current time information is mainly monitored by the one-dimensional data curve (such as the TIC curve), which cannot be observed on the two-dimensional image and is not intuitive enough for operation.
4. Lack of ability to perform destruction of microbubbles in selected specific areas: the current ultrasonic contrast device usually performs the destruction of microbubbles by using full-emission high-voltage sequences; however, if a specific area can be selected for the destruction of microbubbles, the flow of the contrast agent in partial branch vessels can be observed, which is more beneficial to viewing secondary perfusion of this subregion.

Therefore, in order to overcome the above drawbacks, the present disclosure provides an ultrasonic contrast imaging device and method based on the ultrafast plane wave.

### SUMMARY

The present disclosure provides an ultrasonic contrast imaging device and a method based on the ultrafast plane wave, which processes and analyzes echo signals corresponding to different ultrasonic waves by sequentially emitting ultrasonic plane waves of different waveforms, so as to accurately and effectively acquire the contrast imaging. It guarantees the sensitivity of the contrast imaging and also reduces the consumption of the frame frequency, and at the same time, it can synchronously display the time measurement value in the contrast imaging by a two-dimensional image, which provides a diagnostic basis and a reference for the clinical medicine and guarantees the accuracy and reliability of the contrast imaging in case of reducing the consumption of resources.

The present disclosure provides an ultrasonic contrast imaging device based on the ultrafast plane wave, including:
a waveform acquisition module, configured to acquire a plane wave sequence with an emission waveform of [k, 1, 1−k, −1]; separately emit corresponding ultrasonic plane waves according to an emission order based on a preset emission end; and sequentially collect echo signals corresponding to different ultrasonic plane waves after a target duration;
a signal storage module, configured to perform signal processing on the collected echo signals and store the processed echo signals;
a signal transmission and processing module, configured to transmit the stored echo signals to a master computer; superimpose the different echo signals according to weights of different echo signals based on the master computer to obtain a sum signal; and perform beam synthesis processing on the sum signal to obtain a beam synthesis signal; and
an imaging module, configured to perform a modulus calculation and a pseudo-color mapping display for the beam synthesis signal to obtain an ultrasonic contrast image at a target moment.

Preferably, an ultrasonic contrast imaging device based on the ultrafast plane wave is provided, wherein the waveform acquisition module includes: acquiring the plane wave sequence with the emission waveform of [k, 1, 1−k, −1], and taking k, 1, 1−k, −1 as the emission order of emitting the ultrasonic plane waves, where k is a weight ratio of emission amplitudes of the different ultrasonic plane waves and a value range of k is (0, 1).

Preferably, an ultrasonic contrast imaging device based on the ultrafast plane wave is provided, wherein the waveform acquisition module includes:
a waveform emission unit, configured to:
acquire an emission order of the different ultrasonic plane waves; emit an ultrasonic plane wave A with a weight of k in a first emission sequence based on the preset emission end; and collect an echo signal a of the ultrasonic plane wave A after the target duration;
emit an ultrasonic plane wave B with a weight of 1 in a second emission sequence according to the emission order based on the preset emission end, and collect an echo signal b of the ultrasonic plane wave B after the target duration;
emit an ultrasonic plane wave C with a weight of 1−k in a third emission sequence according to the emission order based on the preset emission end, and collect an echo signal c of the ultrasonic plane wave C after the target duration;
emit an ultrasonic plane wave D with a weight of −1 in the fourth emission sequence according to the emission order based on the preset emission end, and collect an echo signal d of the ultrasonic plane wave D after the target duration; and
an echo signal summarization unit, configured to sequentially summarize the echo signal a, echo signal b, echo signal c, and echo signal d collected based on the emission order, so as to obtain a plane wave radio frequency signal [a, b, c, d].

Preferably, an ultrasonic contrast imaging device based on the ultrafast plane wave is provided, wherein the signal storage module includes:
a signal acquisition unit, configured to acquire the collected echo signal a, echo signal b, echo signal c, and echo signal d; set a first target processing parameter for a signal processing component; and perform first signal processing for the echo signal a, echo signal b, echo signal c, and echo signal d according to a first preset processing method based on a setting result, wherein the first preset processing method comprises low noise amplification, analog gain amplification, and filtering;
a signal processing unit, configured to perform second signal processing on the echo signals after the first signal processing based on the second preset processing method, and obtain a target processing signal based on a second signal processing result, wherein the second preset processing method comprises ADC sampling, demodulation with a frequency of f1, low-pass filtering, and signal extraction; and
a signal storage unit, configured to set a target storage area in a memory and store the obtained target processing signal in the target storage area.

Preferably, an ultrasonic contrast imaging device based on the ultrafast plane wave is provided, wherein the signal transmission and processing module includes:
a signal transmission unit, configured to acquire the stored echo signals; adapt a transmission bandwidth; and transmit the stored echo signals to the master computer based on the adapted transmission bandwidth;
a signal superposition unit, configured to acquire a weight of each of the echo signals, and superimpose each of the echo signals based on the weight, so as to obtain a superimposed signal;
a beam synthesis unit, configured to upload the superimposed signal to a GPU, and perform a beam synthesis on the superimposed signal based on a preset beam synthesis method, so as to obtain a beam synthesis signal.

Preferably, an ultrasonic contrast imaging device based on the ultrafast plane wave is provided, wherein the imaging module includes:
a signal modulus calculation unit, configured to acquire the obtained beam synthesis signal; map the beam synthesis signal in a preset coordinate system; and perform a modulus calculation for the beam synthesis signal based on a mapping result;
an imaging unit, configured to:
perform signal truncation and compression for the beam synthesis signal 32 bit obtained by the modulus calculation based on a signal range and map it to 8 bit; real-time monitor whether a user submits a contrast imaging request at the same time; and control a software timer to start when the contrast imaging request is present; and
perform an image conversion for the beam synthesis signal based on a start result, which specifically comprises:

$$img ( timecode , x , y ) = max ( img ( t 1 , x , y ) , img ( t 0 , x , y ) ) ,$$

$$img ( t 1 ) = img ( timecode , x , y ) ,$$

where t1 represents a current moment; t0 represents a previous moment; img represents a collected image; x represents a horizontal coordinate value of the image; and y represents a vertical coordinate value of the image.

Preferably, an ultrasonic contrast imaging device based on the ultrafast plane wave is provided, wherein the imaging unit includes:
a signal processing subunit, configured to:
acquire the obtained 8-bit beam synthesis signal and truncate lower two bits of the 8-bit beam synthesis signal, so as to obtain a 6-bit beam synthesis signal, and map a signal amplitude of the 6-bit beam synthesis signal to a y-axis of an amplitude-time pseudo-color mapping map;
calculate, at the same time, an amplitude change at a position (m, n) in an image frame based on a start time of the software timer, and map it to an x-axis of the amplitude-time pseudo-color mapping map based on amplitude change time; and
obtain a target ultrasonic contrast image based on a mapping result; compare amplitude change values between points in the image at the previous moment to and the current moment t1 based on the target ultrasonic contrast image; reserve, at the same time, a peak value in the amplitude change values obtained after the timer starts; and determine a perfusion process of an intensity variation over time based on the peak value.

Preferably, an ultrasonic contrast imaging device based on the ultrafast plane wave is provided, wherein the signal processing subunit includes:
performing a destruction of contrast microbubbles on a specific vascular branch when the perfusion is finished, wherein the device specifically includes:
a monitoring subunit, configured to monitor an operation instruction in real time, and activate a sampling frame in a target image area when the operation instruction is present;
a position selection subunit, configured to select an interested region based on the sampling frame; interrupt a sequence emission of the ultrasonic contrast based on a selected result; activate a target high voltage to emit a long-period focusing beam to the selected interested region; and display emission parameters of the focusing beam and an adjustable button on a preset display interface at the same time; and
an imaging subunit, configured to continue an emission of an ultrasonic imaging sequence when an emission of the focusing beam is finished, and perform the contrast imaging in real time based on preset ultrasonic contrast imaging steps.

Preferably, an ultrasonic contrast imaging device based on the ultrafast plane wave is provided, including:
a component configuration subunit, configured to set a second target processing parameter for the signal processing component, and perform the first signal processing for the echo signal c based on a setting result;
a signal processing subunit, configured to:
perform second demodulation for the echo signal c after the first signal processing based on a frequency f2, and store the demodulated echo signal c in a memory; and
perform the beam synthesis processing for the stored echo signal c, and perform the modulus calculation and the pseudo-color mapping display for the beam synthesis signal obtained by the beam synthesis processing, so as to obtain an ultrasonic B image of the target moment.

The present disclosure provides an ultrasonic contrast imaging method based on the ultrafast plane wave, including:
step 1, acquiring a plane wave sequence with an emission waveform of [k, 1, 1−k, −1]; separately emitting corresponding ultrasonic plane waves according to an emission order based on a preset emission end; and sequentially collecting echo signals corresponding to different ultrasonic plane waves after a target duration;
step 2, performing signal processing on the collected echo signals and storing the processed echo signals;
step 3, transmitting the stored echo signals to the master computer; superimposing different echo signals according to weights of different echo signals based on the master computer to obtain a sum signal; and performing beam synthesis processing on the sum signal to obtain a beam synthesis signal; and
step 4, performing a modulus calculation and a pseudo-color mapping display for the beam synthesis signal to obtain an ultrasonic contrast image at a target moment.

Compared to the prior art, the present disclosure includes the following beneficial effects.
1. It processes and analyzes echo signals corresponding to different ultrasonic waves by sequentially emitting ultrasonic plane waves of different waveforms, so as to accurately and effectively acquire the contrast imaging. It guarantees the sensitivity of the contrast imaging and also reduces the consumption of the frame frequency, and at the same time, it can synchronously display the time measurement value in the contrast imaging by the two-dimensional image, which provides the diagnostic basis and a reference for the clinical medicine and guarantees the accuracy and reliability of the contrast imaging in case of reducing the consumption of resources.
2. It ensures the accuracy and reliability of the obtained echo signals by processing the collected echo signals; and at the same time, the obtained target processing signal is stored in the memory, which facilitates accurate and effective retrieval of the target processing signal when performing the contrast imaging, so as to guarantee the accuracy and reliability of the contrast imaging.

Other features and advantages of the present disclosure will be illustrated in the subsequent specification, and partial will become apparent from the specification or will be understood by implementing the present disclosure. The objectives and other advantages of the present disclosure may be realized and obtained by structures specifically indicated in the written specification, claims, and drawings.

The technical solutions of the present disclosure are described in further detail below by the drawings and embodiments.

### BRIEF DESCRIPTION OF DRAWINGS

The drawings are used to provide a further understanding of the present disclosure and constitute a part of the specification, and are used in conjunction with the embodiments of the present disclosure to illustrate the present disclosure and do not constitute a limitation of the present disclosure. In the drawings:

FIG. 1 shows a structure diagram of an ultrasonic contrast imaging device based on an ultrafast plane wave in the embodiments of the present disclosure;

FIG. 2 shows a structure diagram of a signal storage module in an ultrasonic contrast imaging device based on the ultrafast plane wave in the embodiments of the present disclosure;

FIG. 3 shows a schematic diagram of a principle of pseudo-color mapping display in an ultrasonic contrast imaging device based on the ultrafast plane wave in the embodiments of the present disclosure; and

FIG. 4 shows a flow diagram of an ultrasonic contrast imaging method based on the ultrafast plane wave in the embodiments of the present disclosure.

### DETAILED DESCRIPTION OF EMBODIMENTS

The preferred embodiments of the present disclosure are described below in conjunction with drawings. It should be understood that the preferred embodiments described herein are intended only to illustrate and explain the present disclosure, and are not intended to limit the present disclosure.

### Embodiment 1

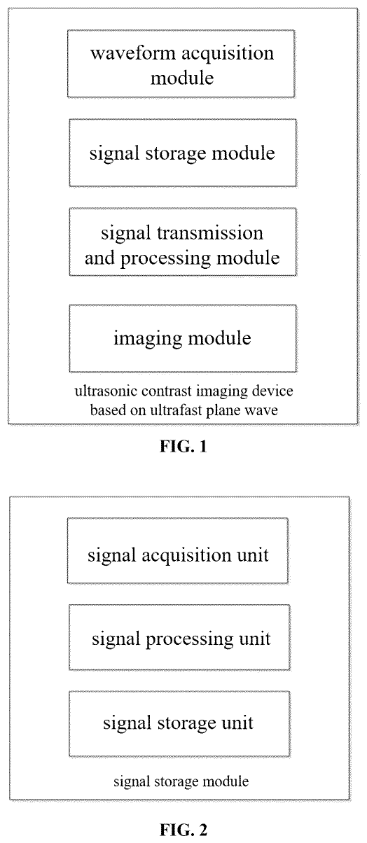

The embodiment provides an ultrasonic contrast imaging device based on the ultrafast plane wave, as shown in FIG. 1, including:
a waveform acquisition module, configured to acquire a plane wave sequence with an emission waveform of [k, 1, 1−k, −1]; separately emit corresponding ultrasonic plane waves according to an emission order based on a preset emission end; and sequentially collect echo signals corresponding to different ultrasonic plane waves after a target duration;
a signal storage module, configured to perform signal processing on the collected echo signals and store the processed echo signals;
a signal transmission and processing module, configured to transmit the stored echo signals to a master computer; superimpose the different echo signals according to weights of different echo signals based on the master computer to obtain a sum signal; and perform beam synthesis processing on the sum signal to obtain a beam synthesis signal; and
an imaging module, configured to perform a modulus calculation and a pseudo-color mapping display for the beam synthesis signal to obtain an ultrasonic contrast image at a target moment.

In the embodiment, the preset emission end, i.e. the front end in the system, is set in advance. For example, it can be an ultrasonic plane wave emitter.

In the embodiment, the emission order refers to emitting based on [k, 1, 1−k, −1], wherein 1 is a normalized amplitude ratio, which is not the only number representing reality.

In the embodiment, the target duration is set in advance, which is used to characterize the length of time needed to wait for emitting the ultrasonic plane wave, so as to accurately and effectively monitor the echo signal, wherein the echo signal is a feedback signal of the ultrasonic plane wave signal after touching a detected object.

In the embodiment, the signal processing refers to signal processing such as ADC sampling, demodulation with a frequency of f1, low-pass filtering, signal extraction after performing low noise amplification, analog gain amplification, and filtering for the echo signal.

In the embodiment, the master computer is set up in advance, which is configured for processing the obtained echo signal, so as to accurately and effectively acquire the contrast imaging.

In the embodiment, the sum signal refers to the signal obtained after superimposing the obtained echo signals, which is used for generating the contrast imaging.

In the embodiment, the beam synthesis processing is the synthesis processing of the sum signal by using a known beam synthesis solution or method, wherein the beam synthesis signal is obtained by performing the beam synthesis for the sum signal.

In the embodiment, the pseudo-color mapping display refers to the processing of assigning color values to grayscale values based on specific criteria.

The above solution includes the following beneficial effects. The device processes and analyzes echo signals corresponding to different ultrasonic waves by sequentially emitting ultrasonic plane waves of different waveforms, so as to accurately and effectively acquire the contrast imaging. It guarantees the sensitivity of the contrast imaging and also reduces the consumption of the frame frequency, and at the same time, it can synchronously display the time measurement value in the contrast imaging by the two-dimensional image, which provides the diagnostic basis and a reference for the clinical medicine and guarantees the accuracy and reliability of the contrast imaging in case of reducing the consumption of resources.

### Embodiment 2

Based on Embodiment 1, the embodiment provides an ultrasonic contrast imaging device based on the ultrafast plane wave, wherein the waveform acquisition module includes: acquiring the plane wave sequence with the emission waveform of [k, 1, 1−k, −1], and taking k, 1, 1−k, −1 as the emission order of emitting the ultrasonic plane waves, where k is a weight ratio of emission amplitudes of the different ultrasonic plane waves and a value range of k is (0, 1).

The above solution includes the following beneficial effects. By emitting the plane wave sequence with different waveforms in sequence, it improves the sensitivity of the contrast imaging and also reduces the consumption of the frame frequency, so as to guarantee the accuracy and reliability of the contrast imaging.

### Embodiment 3

Based on Embodiment 1, the embodiment provides an ultrasonic contrast imaging device based on the ultrafast plane wave, wherein the waveform acquisition module includes:
a waveform emission unit, configured to:
acquire an emission order of the different ultrasonic plane waves; emit an ultrasonic plane wave A with a weight of k in a first emission sequence based on the preset emission end; and collect an echo signal a of the ultrasonic plane wave A after the target duration;
emit an ultrasonic plane wave B with a weight of 1 in a second emission sequence according to the emission order based on the preset emission end, and collect an echo signal b of the ultrasonic plane wave B after the target duration;
emit an ultrasonic plane wave C with a weight of 1−k in a third emission sequence according to the emission order based on the preset emission end, and collect an echo signal c of the ultrasonic plane wave C after the target duration;
emit an ultrasonic plane wave D with a weight of −1 in the fourth emission sequence according to the emission order based on the preset emission end, and collect an echo signal d of the ultrasonic plane wave D after the target duration; and
an echo signal summarization unit, configured to sequentially summarize the echo signal a, echo signal b, echo signal c, and echo signal d collected based on the emission order, so as to obtain a plane wave radio frequency signal [a, b, c, d].

The above solution includes the following beneficial effects. By sequentially emitting the ultrasonic plane waves with different weights, collecting the corresponding echo signals after the target duration, and summarizing the collected echo signals, it provides the convenience and basis for performing the ultrasonic contrast imaging.

### Embodiment 4

Based on Embodiment 1, the embodiment provides an ultrasonic contrast imaging device based on the ultrafast plane wave, as shown in FIG. 2, wherein the signal storage module includes:
a signal acquisition unit, configured to acquire the collected echo signal a, echo signal b, echo signal c, and echo signal d; set a first target processing parameter for a signal processing component; and perform first signal processing for the echo signal a, echo signal b, echo signal c, and echo signal d according to a first preset processing method based on a setting result, wherein the first preset processing method comprises low noise amplification, analog gain amplification, and filtering;
a signal processing unit, configured to perform second signal processing on the echo signals after the first signal processing based on the second preset processing method, and obtain a target processing signal based on the second signal processing result, wherein the second preset processing method comprises ADC sampling, demodulation with a frequency of f1, low-pass filtering, and signal extraction; and
a signal storage unit, configured to set a target storage area in a memory and store the obtained target processing signal in the target storage area.

In the embodiment, the signal processing component is known in advance, for example, it can be a signal amplifier and a filter.

In the embodiment, the first target processing parameter refers to a processing parameter of the signal processing component for the echo signal, which can specifically be the cut-off frequency, the amplifier gain, and the coefficient of the extractor.

In the embodiment, the first preset processing method is set in advance, which specifically can be low noise amplification, analog gain amplification, and filtering in sequence.

In the embodiment, the second preset processing method is set in advance, which specifically can be signal processing such as ADC sampling, demodulation with a frequency of f1, low-pass filtering, and signal extraction.

In the embodiment, the target processing signal refers to the final signals obtained after performing the first signal processing and the second signal processing on the echo signal a, the echo signal b, the echo signal c, and the echo signal d.

In the embodiment, the target storage area is an area in the memory for storing the target processing signal.

The above solution includes the following beneficial effects. It ensures the accuracy and reliability of the obtained echo signals by processing the collected echo signals. At the same time, the obtained target processing signal is stored in the memory, which facilitates the accurate and effective retrieval of the target processing signal when performing the contrast imaging, so as to guarantee the accuracy and reliability of the contrast imaging.

### Embodiment 5

Based on Embodiment 1, the embodiment provides an ultrasonic contrast imaging device based on the ultrafast plane wave, wherein the signal transmission and processing module includes:
a signal transmission unit, configured to acquire the stored echo signals; adapt a transmission bandwidth; and transmit the stored echo signals to the master computer based on the adapted transmission bandwidth;
a signal superposition unit, configured to acquire a weight of each of the echo signals, and superimpose each of the echo signals based on the weight, so as to obtain a superimposed signal;
a beam synthesis unit, configured to upload the superimposed signal to a GPU, and perform a beam synthesis on the superimposed signal based on a preset beam synthesis method, so as to obtain a beam synthesis signal.

In the embodiment, adapting the transmission bandwidth refers to adjusting the transmission bandwidth to 4G/s and above.

In the embodiment, the GPU is set in advance, which specifically refers to a graphics processing unit.

In the embodiment, the preset beam synthesis method is known in advance, i.e., the prior art method, wherein the corresponding beam synthesizing operation is performed by this method.

The above solution includes the following beneficial effects. By adapting the transmission bandwidth, uploading each echo signal to the master computer by the adapted transmission bandwidth; and performing the corresponding beam synthesis operation in the master computer, it provides convenience and guarantee for obtaining the ultrasonic contrast imaging, so as to guarantee the accuracy and reliability of the contrast ultrasonic contrast.

### Embodiment 6

Based on Embodiment 1, the embodiment provides an ultrasonic contrast imaging device based on the ultrafast plane wave, wherein the imaging module includes:
a signal modulus calculation unit, configured to acquire the obtained beam synthesis signal; map the beam synthesis signal in a preset coordinate system; and perform a modulus calculation for the beam synthesis signal based on a mapping result;
an imaging unit, configured to:
perform signal truncation and compression for the beam synthesis signal 32 bit obtained by the modulus calculation based on a signal range and map it to 8 bit; real-time monitor whether a user submits a contrast imaging request at the same time; and control a software timer to start when the contrast imaging request is present; and
perform an image conversion for the beam synthesis signal based on a start result, which specifically comprises:

$$img ( timecode , x , y ) = max ( img ( t 1 , x , y ) , img ( t 0 , x , y ) ) ,$$

$$img ( t 1 ) = img ( timecode , x , y ) ,$$

where t1 represents the current moment; t0 represents the previous moment; img represents the collected image; x represents the horizontal coordinate value of the image; and y represents the vertical coordinate value of the image.

In the embodiment, the preset coordinate system is set in advance, which is used for displaying the obtained beam synthesis signal in the preset coordinate system so as to perform the modulus calculation accurately and effectively for the beam synthesis signal.

In the embodiment, the modulus can be obtained by calculating according to the modulus calculation formula based on the length of the beam synthesis signal in the preset in the coordinate system.

In the embodiment, the contrast imaging request is submitted by the user clicking in the system, which is used to characterize the need for contrast imaging.

The above solution includes the following beneficial effects. By performing the modulus calculation and image conversion for the obtained beam synthesis signal, it provides the convenience and basis for the contrast imaging finally obtained so as to ensure the accuracy and reliability of the contrast imaging finally obtained.

### Embodiment 7

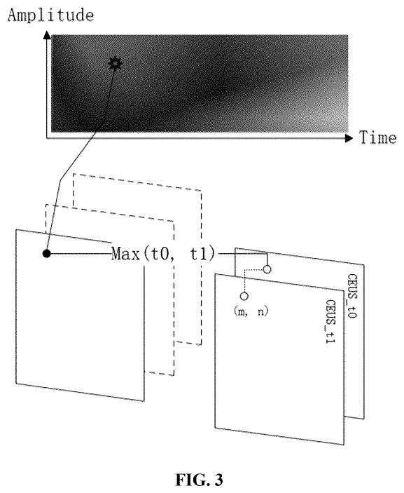

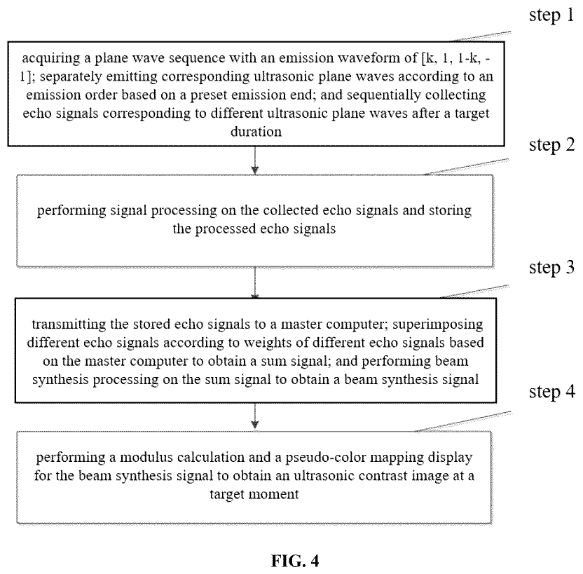

Based on Embodiment 6, the embodiment provides an ultrasonic contrast imaging device based on the ultrafast plane wave, as shown in FIG. 3, wherein the imaging unit includes:
a signal processing subunit, configured to:
acquire the obtained 8-bit beam synthesis signal and truncate the lower two bits of the 8-bit beam synthesis signal so as to obtain a 6-bit beam synthesis signal, and map the signal amplitude of the 6-bit beam synthesis signal to the y-axis of the amplitude-time pseudo-color mapping map;
calculate, at the same time, an amplitude change at a position (m, n) in an image frame based on a start time of the software timer, and map it to an x-axis of the amplitude-time pseudo-color mapping map based on amplitude change time; and
obtain a target ultrasonic contrast image based on a mapping result; compare amplitude change values between points in the image at the previous moment to and the current moment t1 based on the target ultrasonic contrast image; reserve, at the same time, a peak value in the amplitude change values obtained after the timer starts; and determine a perfusion process of an intensity variation over time based on the peak value.

In the embodiment, the target ultrasonic contrast image refers to a contrast image that integrates the intensity and the time, obtained after mapping the signal amplitude and the time measurement value into the contrast image.

In the embodiment, the peak value refers to the maximum value in the amplitude change values between points.

In the embodiment, determining the perfusion process of the intensity variation over time based on the peak value refers to determining a perfusion process of the contrast agent according to the change condition of the peak value, so as to ensure the accuracy and reliability of the contrast imaging finally obtained.

The above solution includes the following beneficial effects. By processing the beam synthesis signal, the signal amplitude and the time information corresponding to the signal amplitude of the beam synthesis signal are mapped in the contrast imaging, which accurately and effectively acquires the final required contrast imaging and provides a reliable basis for monitoring the perfusion process.

### Embodiment 8

Based on Embodiment 7, the embodiment provides an ultrasonic contrast imaging device based on the ultrafast plane wave, wherein the signal processing subunit includes:
performing a destruction of contrast microbubbles on a specific vascular branch when the perfusion is finished, wherein the device specifically includes:
a monitoring subunit, configured to monitor an operation instruction in real time, and activate a sampling frame in a target image area when the operation instruction is present;
a position selection subunit, configured to select an interested region based on the sampling frame; interrupt a sequence emission of the ultrasonic contrast based on a selected result; activate a target high voltage to emit a long-period focusing beam to the selected interested region; and display emission parameters of the focusing beam and an adjustable button on a preset display interface at the same time; and
an imaging subunit, configured to continue an emission of an ultrasonic imaging sequence when an emission of the focusing beam is finished, and perform the contrast imaging in real time based on preset ultrasonic contrast imaging steps.

In the embodiment, the contrast microbubbles refer to small bubbles that appear in the process of perfusing the contrast agent.

In the embodiment, the target image refers to contrast imaging.

In the embodiment, the interested region refers to a region where contrast microbubbles are present in the contrast imaging.

In the embodiment, the target high voltage is known in advance.

In the embodiment, the adjustable button refers to a button that can adjust the emission parameters of the focusing beam.

In the embodiment, the preset display interface is set in advance.

In the embodiment, the preset ultrasonic contrast imaging steps are all of the execution steps in the above contrast imaging.

The above solution includes the following beneficial effects. By destructing the contrast microbubbles existing in the contrast agent perfusion process according to the obtained contrast imaging, it improves the accuracy of the destruction of the contrast microbubbles and also guarantees the accuracy and the reliability of the final obtained contrast imaging.

### Embodiment 9

Based on Embodiment 3, the embodiment provides an ultrasonic contrast imaging device based on the ultrafast plane wave, wherein the waveform emission unit includes:
a component configuration subunit, configured to set a second target processing parameter for the signal processing component, and perform first signal processing for the echo signal c based on a setting result;
a signal processing subunit, configured to:
perform second demodulation for the echo signal c after the first signal processing based on a frequency f2, and store the demodulated echo signal c in a memory; and
perform the beam synthesis processing for the stored echo signal c, and perform the modulus calculation and the pseudo-color mapping display for the beam synthesis signal obtained by the beam synthesis processing, so as to obtain an ultrasonic B image of the target moment.

In the embodiment, the second target processing parameter refers to a parameter different from the first target processing parameter, which is set for the purpose of processing the echo signal c individually, so as to effectively acquire the ultrasonic B image at the target moment.

The above solution includes the following beneficial effects. By setting different target processing parameters for the signal processing component and processing the echo signal c based on the set signal processor, and performing the contrast imaging after the second demodulation for the echo signal c after the first signal processing by frequency f2 at the same time, it accurately and effectively acquires the ultrasonic B image at the target moment.

### Embodiment 10

The embodiment provides an ultrasonic contrast imaging method based on the ultrafast plane wave, as shown in FIG. 4, including the following steps.
Step 1: acquiring a plane wave sequence with an emission waveform of [k, 1, 1−k, −1]; separately emitting corresponding ultrasonic plane waves according to an emission order based on a preset emission end; and sequentially collecting echo signals corresponding to different ultrasonic plane waves after a target duration.
Step 2: performing signal processing on the collected echo signals and storing the processed echo signals.
Step 3: transmitting the stored echo signals to the master computer; superimposing different echo signals according to weights of different echo signals based on the master computer to obtain a sum signal; and performing beam synthesis processing on the sum signal to obtain a beam synthesis signal.
Step 4: performing a modulus calculation and a pseudo-color mapping display for the beam synthesis signal to obtain an ultrasonic contrast image at a target moment.

The above solution includes the following beneficial effects. It processes and analyzes echo signals corresponding to different ultrasonic waves by sequentially emitting ultrasonic plane waves of different waveforms, so as to accurately and effectively acquire the contrast imaging. It guarantees the sensitivity of the contrast imaging and also reduces the consumption of the frame frequency, and at the same time, it can synchronously display the time measurement value in the contrast imaging by the two-dimensional image, which provides the diagnostic basis and a reference for the clinical medicine, and guarantees the accuracy and reliability of the contrast imaging in case of reducing the consumption of resources.

Obviously, those skilled in the art can make various changes and variations to the present disclosure without departing from the spirit and scope of the present disclosure. Therefore, the present disclosure is intended to include these modifications and variations if these modifications and variations fall within the scope of the claims of the present disclosure and their technical equivalents.

1. An ultrasonic contrast imaging device based on an ultrafast plane wave, comprising:
a waveform acquisition module, configured to acquire a plane wave sequence with an emission waveform of [k, 1, 1−k, −1]; separately emit corresponding ultrasonic plane waves according to an emission order based on a preset emission end; and sequentially collect echo signals corresponding to different ultrasonic plane waves after a target duration;
a signal storage module, configured to perform signal processing on the collected echo signals and store the processed echo signals;
a signal transmission and processing module, configured to transmit the stored echo signals to a master computer; superimpose the different echo signals according to weights of different echo signals based on the master computer to obtain a sum signal; and perform beam synthesis processing on the sum signal to obtain a beam synthesis signal; and
an imaging module, configured to perform a modulus calculation and a pseudo-color mapping display for the beam synthesis signal to obtain an ultrasonic contrast image at a target moment.

2. The ultrasonic contrast imaging device based on the ultrafast plane wave according to claim 1, wherein the waveform acquisition module comprises: acquiring the plane wave sequence with the emission waveform of [k, 1, 1−k, −1], and taking k, 1, 1−k, −1 as the emission order of emitting the ultrasonic plane waves, where k is a weight ratio of emission amplitudes of the different ultrasonic plane waves and a value range of k is (0, 1).

3. The ultrasonic contrast imaging device based on the ultrafast plane wave according to claim 1, wherein the waveform acquisition module comprises:
a waveform emission unit, configured to:
acquire an emission order of the different ultrasonic plane waves; emit an ultrasonic plane wave A with a weight of k in a first emission sequence based on the preset emission end; and
collect an echo signal a of the ultrasonic plane wave A after the target duration;
emit an ultrasonic plane wave B with a weight of 1 in a second emission sequence according to the emission order based on the preset emission end, and collect an echo signal b of the ultrasonic plane wave B after the target duration;
emit an ultrasonic plane wave C with a weight of 1−k in a third emission sequence according to the emission order based on the preset emission end, and collect an echo signal c of the ultrasonic plane wave C after the target duration;
emit an ultrasonic plane wave D with a weight of −1 in a fourth emission sequence according to the emission order based on the preset emission end, and collect an echo signal d of the ultrasonic plane wave D after the target duration; and
an echo signal summarization unit, configured to sequentially summarize the echo signal a, echo signal b, echo signal c, and echo signal d collected based on the emission order, so as to obtain a plane wave radio frequency signal [a, b, c, d].

4. The ultrasonic contrast imaging device based on the ultrafast plane wave according to claim 1, wherein the signal storage module comprises:
a signal acquisition unit, configured to acquire the collected echo signal a, echo signal b, echo signal c, and echo signal d; set a first target processing parameter for a signal processing component; and perform first signal processing for the echo signal a, echo signal b, echo signal c, and echo signal d according to a first preset processing method based on a setting result, wherein the first preset processing method comprises low noise amplification, analog gain amplification, and filtering;
a signal processing unit, configured to perform second signal processing on the echo signals after the first signal processing based on a second preset processing method, and obtain a target processing signal based on a second signal processing result, wherein the second preset processing method comprises ADC sampling, demodulation with a frequency of f1, low-pass filtering, and signal extraction; and
a signal storage unit, configured to set a target storage area in a memory and store the obtained target processing signal in the target storage area.

5. The ultrasonic contrast imaging device based on the ultrafast plane wave according to claim 1, wherein the signal transmission and processing module comprises:
a signal transmission unit, configured to acquire the stored echo signals; adapt a transmission bandwidth; and transmit the stored echo signals to the master computer based on the adapted transmission bandwidth;
a signal superposition unit, configured to acquire a weight of each of the echo signals, and superimpose each of the echo signals based on the weight, so as to obtain a superimposed signal;
a beam synthesis unit, configured to upload the superimposed signal to a GPU, and perform a beam synthesis on the superimposed signal based on a preset beam synthesis method, so as to obtain a beam synthesis signal.

6. The ultrasonic contrast imaging device based on the ultrafast plane wave according to claim 1, wherein the imaging module comprises:
a signal modulus calculation unit, configured to acquire the obtained beam synthesis signal; map the beam synthesis signal in a preset coordinate system; and perform a modulus calculation for the beam synthesis signal based on a mapping result;
an imaging unit, configured to:
perform signal truncation and compression on the beam synthesis signal 32 bit obtained by the modulus calculation based on a signal range and map it to 8 bit; real-time monitor whether a user submits a contrast imaging request at the same time; and control a software timer to start when the contrast imaging request is present; and
perform an image conversion on the beam synthesis signal based on a start result, which specifically comprises:
$$img ( timecode , x , y ) = max ( img ( t 1 , x , y ) , img ( t 0 , x , y ) ) ,$$
$$img ( t 1 ) = img ( timecode , x , y ) ,$$
where t1 represents a current moment; t0 represents a previous moment; img represents a collected image; x represents a horizontal coordinate value of the image; and y represents a vertical coordinate value of the image.

7. The ultrasonic contrast imaging device based on the ultrafast plane wave according to claim 6, wherein the imaging unit comprises:
a signal processing subunit, configured to:
acquire the obtained 8-bit beam synthesis signal and truncate lower two bits of the 8-bit beam synthesis signal so as to obtain a 6-bit beam synthesis signal, and map a signal amplitude of the 6-bit beam synthesis signal to a y-axis of an amplitude-time pseudo-color mapping map;
calculate, at the same time, an amplitude change at a position (m, n) in an image frame based on a start time of the software timer, and map it to an x-axis of the amplitude-time pseudo-color mapping map based on amplitude change time; and
obtain a target ultrasonic contrast image based on a mapping result; compare amplitude change values between points in the image at the previous moment to and the current moment t1 based on the target ultrasonic contrast image; reserve, at the same time, a peak value in the amplitude change values obtained after the timer starts; and determine a perfusion process of an intensity variation over time based on the peak value.

8. The ultrasonic contrast imaging device based on the ultrafast plane wave according to claim 7, wherein the signal processing subunit comprises:
performing a destruction of contrast microbubbles on a specific vascular branch when the perfusion is finished, wherein the device specifically comprises:
a monitoring subunit, configured to monitor an operation instruction in real time, and activate a sampling frame in a target image area when the operation instruction is present;
a position selection subunit, configured to select an interested region based on the sampling frame; interrupt a sequence emission of the ultrasonic contrast based on a selected result; activate a target high voltage to emit a long-period focusing beam to the selected interested region; and
display emission parameters of the focusing beam and an adjustable button on a preset display interface at the same time; and
an imaging subunit, configured to continue an emission of an ultrasonic imaging sequence when the emission of the focusing beam is finished, and perform the contrast imaging in real time based on preset ultrasonic contrast imaging steps.

9. The ultrasonic contrast imaging device based on the ultrafast plane wave according to claim 3, wherein the waveform emission unit comprises:
a component configuration subunit, configured to set a second target processing parameter for the signal processing component, and perform the first signal processing for the echo signal c based on a setting result;
a signal processing subunit, configured to:
perform second demodulation for the echo signal c after the first signal processing based on a frequency f2, and store the demodulated echo signal c in a memory; and
perform the beam synthesis processing for the stored echo signal c, and perform the modulus calculation and the pseudo-color mapping display for the beam synthesis signal obtained by the beam synthesis processing, so as to obtain an ultrasonic B image of the target moment.

10. An ultrasonic contrast imaging method based on an ultrafast plane wave, comprising:
step 1, acquiring a plane wave sequence with an emission waveform of [k, 1, 1−k, −1]; separately emitting corresponding ultrasonic plane waves according to an emission order based on a preset emission end; and sequentially collecting echo signals corresponding to different ultrasonic plane waves after a target duration;
step 2, performing signal processing on the collected echo signals and storing the processed echo signals;
step 3, transmitting the stored echo signals to a master computer; superimposing the different echo signals according to weights of different echo signals based on the master computer to obtain a sum signal; and performing beam synthesis processing on the sum signal to obtain a beam synthesis signal; and
step 4, performing a modulus calculation and a pseudo-color mapping display for the beam synthesis signal to obtain an ultrasonic contrast image at a target moment.
